(19) United States
(12) Patent Application Publication
(10) Pub. No.: US 2003/0175852 A1
(43) Pub. Date: Sep. 18, 2003

(54) Ehancement of in situ hybridization

(76) Inventors: Krishan L Kalra, Danville, CA (US); Qian-Shu Wang, San Ramon, CA (US); Jia Kuan Jin, San Francisco, CA (US)

Correspondence Address: The Law Offices Of James C Weseman, Suite 1600, 401 West A Street, San Diego, CA, 92101, US

(21) Appl. No.: 10/381,159

(22) PCT Filed: Sep. 17, 2001
(86) PCT No.: PCT/US01/29598

(30) Foreign Application Priority Data
Sep. 15, 2000 (US) 60232671

(51) Int. Cl. G01N 1/30; G01N 33/48

(52) U.S. Cl. 435/40.5

(57) ABSTRACT

Methods and compositions for improving in situ hybridization analysis of aldehyde fixed tissue are described.

### TECHNICAL FIELD

The invention concerns in situ hybridization staining of aldehyde-fixed and embedded tissue sections.

### BACKGROUND OF THE INVENTION

Tissue sections obtained from clinical specimens or animal experimentation frequently are fixed, embedded, and stored in a form suitable for later examination by light microscopy. Such embedding processes generally include the well known steps of specimen fixation, dehydration, clearing, paraffin infiltration or impregnation, blocking or embedding in a block of paraffin, slicing the block and specimen into thin sections, mounting the sections on slides, removing the paraffin and solvents employed for this purpose (deparaffinizing), and staining the sections prior to microscopic analysis. Histochemical staining reagents, especially monoclonal antibody and nucleic acid probe reagents, currently permit examination of at least certain of these fixed tissue samples for the presence of particular antigenic compounds or hybridization sites. Such antigens and sites of interest may be associated with a disease process or pathology, or may identify a particular cell type or tissue. In the case of recently prepared biopsy and autopsy samples, such histochemical analyses are of immediate diagnostic value.

In order to analyze such tissue specimens, it is first deemed desirable to remove a certain portion of the embedding medium, in order to properly expose the tissue to the histochemical staining reagents. Paraffin has been used for many years as an embedding medium in the preparation of tissue specimens for sectioning in a microtome to produce specimen sections for histological studies. The primary purpose of the embedding medium is to permit the specimens to be sectioned and mounted in the natural state. Plastic resins have also been used as embedding medium to provide a harder specimen that allows cutting of thinner sections. However, the use of paraffin-embedding has the advantage that the wax can be dissolved away from specimens prior to staining, allowing sections to be stained in the form of naked slabs of biopolymer and avoiding the extra difficulties and artifacts associated with the presence of unremovable resin-embedding medium.

Recent improvements in paraffin-embedding compositions broaden its applicability while maintaining its compatibility with downstream manipulation and analysis of samples. For example, an improve paraffin-based embedding material, which includes a mixture of paraffin and an effective amount of ethylene-vinyl acetate copolymer (0.5% to 5% by weight of paraffin) allows shorter infiltration time and thinner sections (U.S. Pat. No. 4,497,792). Another improvement, the double-embedding technique, yields sections of thin tissue membranes, such as rodent mesenteric membranes that usually measure only 10 microns in thickness. In this method several membranes are fixed and mounted on four needles located at the bottom of a plastic box and then embedded in agarose. The agarose block is removed, dehydrated in alcohol, cleared with HistoPetrol (trade name for a mixture of isoparaffin hydrocarbons), permeated with paraffin and sectioned. The observed tissue morphology is comparable to that obtained with methacrylate plastic embedding but is less time-consuming, less hazardous since no plastic hardener and activator are used, and makes histochemical studies easier.

Consequently, deparaffinization of fixed, e.g. formalin fixed, paraffin embedded tissue sections is still a widely used methodology, particularly in hospital histopathology laboratories for diagnostic purposes.

Xylene, which is a flammable, volatile and toxic organic solvent, is currently commonly used in protocols to solubilize paraffin for deparaffinization of specimen sections. Typically, the microscope slide-mounted specimen is immersed in a xylene bath until the paraffin is solubilized. The deparaffinized specimen is then washed with a series of alcohol solutions of decreasing alcohol concentration, typically as baths in which the specimen is immersed, to remove xylene before a final wash with water. Efforts have been made to replace xylene in the deparaffinization process with less toxic and less volatile solvents. Terpene oil (e.g. available under the trade name AmeriClear from Baxter Health Care Diagnostics, Inc. McGaw Park, Ill.) and isoparaffinic hydrocarbons (e.g. available under the trade name Micro-Clear from Micron Diagnostics, Inc., Fairfax, Va.) produced equal deparaffinization compared to xylene (Jones et al. 1993). However, a series of alcohol washes were still required to remove either solvent prior to the water wash to achieve compatibility with most types of staining, particularly histochemical staining. Furthermore, the use of paraffin-embedded specimens with automated systems, such as staining apparatus, is increasing.

Accordingly, there remains a need for deparaffinization compositions and methods that can effectively remove paraffin or improved paraffin-based embedding materials from specimens prior to histochemical or other diagnostic analyses, while minimizing danger to users, allowing compatibility with automated systems, and maintaining compatibility with downstream analyses. Deparaffinization compositions and methods that entail no or limited toxicity or carcinogenicity, produce no or minimal odors, reduce the quantity of toxic solvents used, minimize hazardous wastes, and/or decrease corrosiveness and flammability are needed.

However, even after deparaffinization, histochemical analyses of tissue specimens have been hampered because of antigenic and hybridization site loss during specimen fixation. Traditional fixation methods frequently have employed aldehyde fixatives, which fix the tissue by causing cross-linking reactions within and between tissue proteins, which can alter or obscure various structures of interest.

Two types of cross-linking reactions have been recognized. The first is a Schiff's base-type polymerization: Formaldehyde condenses with the amino groups of the protein, resulting in the Schiff's base intermediate, which is capable of undergoing rapid polymerization leading to cross-linking of the proteins.

In the second type of reaction, called the Mannich reaction, the formaldehyde can react with both an amino group and an active hydrogen group, resulting in the formation of a Mannich base. Polymerization of the Mannich bases results in protein cross-linking.

Cross-links preserve tissue morphology and integrity, harden the tissue for slicing, and inhibit microbial attack. Unfortunately, the cross-linking process also causes loss of native tissue structure, a result that impedes the usefulness of histochemical staining reagents on tissues fixed with aldehyde reagents such as formaldehyde. The chemistry of the cross-linking of amino acids and proteins by formaldehyde is described in Harlan and Feairheller, “Chemistry of the Cross-Linking of Collagen During Tanning,” and Kelly, et al., “Cross-Linking of Amino Acids By Formaldehyde,” (1976). The role of Mannich-type reactions in cross-linking of protein amino groups and aromatic amino acids with formaldehyde is discussed in Fraenkel-Conrat, et al., J Biol. Chem. (1947) 168:99-118, and Fraenkel-Conrat and Olcott, J Biol. Chem. (1948) 174:827-843. Further discussions of aldehyde cross-linking reactions are found in Fox, J. Histochem. Cytochem. (1985) 33:845-855; Jones, “Reactions of aldehyde with unsaturated fatty acids during histological fixation,” in Fixation in Histochemistry, P. J. Stoward, ed. (1973); and Kunkel et al., Mol. Cell. Biochem. (1981) 34:3. Mannich type reactions are described in general in March, “Advanced Organic Chemistry,” particularly at 333, 424, 670-672 (1968).

In an attempt to circumvent the disadvantages of aldehyde fixation, alternative fixation methods have been developed, such as microwave heating (Mayers, J. Clin. Pathol. (1970) 28:273; Hopwood et al., Histochem. J. (1984) 16:1171) and alcohol immersion (Battifora and Kopinski, J. Histochem. Cytochem. (1986) 34:1095). Despite some advantages of alternative fixation methods, they have not displaced aldehyde fixation in general use. Their limited acceptance may reflect drawbacks present in these alternative methods. For example, microwave heating lyses red cells and disrupts membrane lipids. Although ethanol fixation is reported to produce improved antigenicity of tissue samples, ethanol causes increased cellular shrinkage (Battifora and Kopinski). Consequently, methods for restoring fidelity to aldehyde fixed tissues continue to be useful for specimens generated by current clinical practices.

In addition, methods for restoring tissue fidelity are useful because of the vast number of aldehyde fixed tissue samples already in collections. These stored tissue samples provide a rich reservoir of material for retrospective histochemical examination. If a suitable method of subsequent histochemical staining were available, newly generated histochemical data could be combined with existing diagnostic results obtained from traditional investigations on the same tissues. Often clinical samples are saved for decades, so that the clinical outcome of the patient's underlying pathological process already is known. In the case of experimental tissues, such as those obtained from animals in toxicology testing, other measurements of pathology and toxicity in general already will have been performed and documented. In both cases, histochemical analyses of the affected tissues could add important correlative information.

Because of the development of histochemical reagents over the past decades, analyses can now be performed that were impossible at the time many tissues were originally stored. In addition, new knowledge or hypotheses concerning the disease process may prompt reexamination of stored tissues. Histochemical studies on stored tissue samples provide a relatively time- and cost-effective means for performing a clinical study on a statistically large sample population. Therefore the application of histochemical analyses to routine clinically- or experimentally-derived embedded tissue sections is a matter of considerable interest.

A procedure for restoring antigenicity of formalin-fixed, paraffin embedded tissue sections by heating the tissue in a microwave in a heavy metal solution has been described in Shi, et al., J. Histochem. Cytochem. 39(b):74148 (1991). This procedure provides enhanced immunostaining in approximately three-fourths of the samples tested. The described method is part of a process that involves the steps of tissue section deparaffinization and rehydration, brief treatment with aqueous peroxide to block endogenous peroxidase, washing of the slides with distilled water, covering the slides with distilled water or a heavy metal solution, and brief microwave heating for several minutes. Following this procedure, slides are cooled, rinsed, and immunostained in a conventional fashion.

This procedure for restoring antigenicity is subject to certain limitations. First, it requires the use of a microwave oven to heat the tissue samples. Many laboratories may not be equipped with a microwave oven, and some tissue samples may not be suited to microwave heating. A need exists for an antigen retrieval method that can be used at room temperature, without any external heat source.

In addition, the previously described procedure is especially suitable for tissues embedded in a hydrocarbon medium such as paraffin. It is not well suited for tissue sections embedded in celloidin, a preferred embedding medium for bony tissues. A need also exists for a method that is suitable for use with celloidin embedded tissues. Moreover, a need particularly exists for a method that may be used with decalcified bony tissue samples, since decalcified tissues are often refractory to the previously described procedure.

### DISCLOSURE OF THE INVENTION

The present invention provides methods and kits for improved in situ hybridization reactions that result in increased ease of use, easier adaptation to automated tissue specimen processing, and increased sensitivity and histochemical reactivity between the nucleic acid probe and the tissue specimen.

In one aspect the present invention provides a method for improving the histochemical reactivity of a tissue specimen fixed with an aldehyde fixing agent and embedded in an embedding medium comprising contacting the tissue specimen with a solution comprising a dewaxing solvent composition and an aldehyde releasing reagent composition. The solution and the tissue specimen are then heated and the heating is maintained for a time sufficient to solubilize at least a portion of the embedding medium and to release at least a portion of the aldehyde bonds fixing the tissue specimen. Thereafter, a histochemical reaction will be performed on the tissue specimen.

Other aspects of the invention include methods that combine the dewaxing and releasing method with in situ hybridization techniques and kits of reagents and solutions for performing the present methods.

### DETAILED DESCRIPTION OF THE INVENTION

The present invention provides methods and kits for improved in situ hybridization reactions that result in increased ease of use, easier adaptation to automated tissue specimen processing, and increased sensitivity and histochemical reactivity between the nucleic acid probe and the tissue specimen.

In one aspect the present invention provides a method for improving the histochemical reactivity of a tissue specimen fixed with an aldehyde fixing agent and embedded in an embedding medium comprising contacting the tissue specimen with a solution comprising a dewaxing solvent composition and an aldehyde releasing reagent composition. The solution and the tissue specimen are then heated and the heating is maintained for a time sufficient to solubilize at least a portion of the embedding medium and to release at least a portion of the aldehyde bonds fixing the tissue specimen. Thereafter, a histochemical reaction will be performed on the tissue specimen.

In an in situ hybridization (ISH) assay, microwave heating of paraformaldehyde- or formalin-fixed tissues has been shown to enhance hybridization signals and reduce background compared to conventional protease digestion, and several microwave pretreatment systems have been reported (1-5). These pretreatment systems, however, require deparaffininization of paraffin-embedded tissues before microwave heating is applied. The invention described here offers deparaffinization and an improvement of hybridization signals in one step using a specially designed solution, Nucleic Acid Retrieval Solution I (NAR-I), by microwave heating.

The present invention minimizes or eliminates the use of xylene solvents in histological laboratories. The compositions and methodology described herein effectively remove paraffin or other wax residue from tissue sections and have no adverse effect on the quality or histological reactivity of tissue sections prepared for immunohistochemistry and in situ hybridization. Application of this methodology can be extended to other analytical applications where removal of embedding medium from tissue sections are desired, such as in situ hybridization, classical dye stains and special stains.

By “embedding medium” is meant any composition that is solid at room temperature and is used in the histochemical art for embedding or otherwise supporting reactive tissue specimens for histochemical or other analyses, such as in situ hybridization, special stains and classical dye stains. As one example of an embedding medium, wax is often used for this purpose.

By “wax” is meant a composition used in the histochemical art for embedding reactive specimens for histochemical or other analyses that is solid at room temperature, usually consists of a complex mixture of higher hydrocarbons often including esters of higher fatty acids and higher glycols, may be mineral, natural or synthetic in origin, is harder and more brittle than fats, is soluble in oils and fats, and can optionally contain additives that enhance its specimen-embedding properties. Paraffin is an example of a mineral wax most commonly used in the histochemical field. Paraffin is typically prepared by distillation of petroleum, and is a mixture of primarily solid saturated hydrocarbons.

By “histochemical” is meant to include the techniques and methods known as immunohistochemical, cytochemical, histopathologic, enzyme histochemical, special stain, micro technique, in situ hybridization, and the use of molecular probes. Texts illustrating histochemical techniques include “Histochemical and Immnunochemical techniques: Application to pharmacology and toxicology,” (1991) Bach, P. and Baker, J., eds., Chapman & Hall, New York, N.Y., pp. 1-9, and in “Stains and Cytochemical Methods,” (1993) M. A. Hayat, ed., Plenum Press, New York, N.Y.

By “removing the embedding medium” is meant removing a sufficient amount of the embedding medium so as to permit the reactive tissue specimen to be subjected to analysis. Typically, such analysis is histochemical, and the amount of the embedding medium that should be removed will be the amount sufficient to permit the analysis technique of choice to gain access to at least one of the reactive sites in the reactive tissue specimen.

By “reactive tissue specimen” is meant a sample of animal or plant cells or tissues which is selected and treated so as to preserve a detectable amount of the native histological reactivity inherent in the sampled organism prior to the sampling. Typically, such specimens, are obtained as tissue sections by biopsy, necropsy and the like, all in accordance with techniques well know in the histochemical arts.

Because the present compositions are typically prepared by combining components without a precise determination of the final volume of the composition or accounting for volume changes upon mixing, the percentages for each component are qualified with the term “about” or “approximately”, with the understanding that one skilled in the art would appreciate the imprecision of the values as a consequence of composition preparation; however, preferably percentage values are taken to mean their precise value when volume changes upon mixing are accounted for.

Dewaxing Solvent Compositions

In one aspect, the present invention employs dewaxing solvent compositions for removing embedding media, and particularly wax or modified wax-based embedding media, particularly paraffin or paraffin-based, from tissue specimens prior to histochemical or other analyses, while minimizing danger to users, allowing compatibility with automated use, and maintaining compatibility with downstream analyses. In this regard, it is considered important to remove a portion of the embedding medium associated with the tissue specimen without substantial adverse effect on the histological reactivity of the specimen. In further embodiments the composition of the invention may optionally be diluted with water.

The present dewaxing solvent compositions comprise a number of separate components, including a non-polar organic solvent, a polar organic solvent and a surfactant in amounts sufficient to release a sufficient portion of the embedding medium associated with the tissue specimen to permit histochemical analysis without substantial adverse effect on the histochemical reactivity of the tissue specimen.

The non-polar organic solvent is a hydrocarbon or mixture of hydrocarbons (e.g. as from a petroleum distillate) that has a boiling point well above room temperature, preferably above 110° C., more preferably from about 140° C. to about 250° C., that is in liquid phase at the temperatures used with the present invention (usually 5° to 50° C.), and that is capable of dissolving paraffin used for embedding biological specimens. The non-polar solvent can be a complex mixture of long-chain linear and branches alkane hydrocarbons containing for example esters of fatty acids and higher glycols. As a representative example for removing an embedding medium, the paraffin solubility of the solvent at 25° C. is typically at least 0.1 gram paraffin per liter of solvent, preferably 0.1 gram per 100 mL of solvent, more preferably 0.1 gram per 10 mL of solvent, and most preferably capable of a dissolving an amount of paraffin equal to about 50% of the solvent solutions weight. The non-polar solvent is further miscible with a polar organic solvent when used in a composition of the invention. Examples of non-polar organic solvents include aromatic hydrocarbons, aliphatic hydrocarbons, terpenes, other oils, and petroleum distillates. Preferred non-polar organic solvents have little or no toxic effects. Furthermore preferred solvents are those not classified by the Environmental Protection Agency as hazardous waste. A preferred non-polar solvent furthermore has a flash point higher than about 60° C., which minimizes flammability. A preferred solvent furthermore lacks toxicity, carcinogenicity, and corrosiveness. An isoparaffinic hydrocarbon is an example of a preferred non-polar solvent, in part because of its lack of toxicity, carcinogenicity, corrosiveness and flammability (Mullin et al. 1990). Preferred isoparaffins are branched aliphatic hydrocarbons with a carbon skeleton length ranging from approximately C10 to C15, or mixtures thereof.

One preferred isoparaffin hydrocarbon mixture has a flashpoint of about 74° C. Another preferred non-polar solvent is a mixture of C10 to C50 branched or linear hydrocarbon chains having a distillation range from a boiling point of 150° C. to about 250° C., and has the general formula of CnH(2n±m) where n=10−50 and m=0−4. Mineral spirits is another preferred non-polar organic solvent. A preferred terpene is limonene. Other terpenes that can be used include terpenes, terpinenes and terpineols. Less preferably the solvent is an aromatic hydrocarbon solvent such as an alkyl benzene, e.g. xylene, or a dialkylbenzene, e.g. toluene. Toluene and xylene are less preferred because of their toxicity and rating as hazardous waste. Furthermore, as discussed below, even when xylene or toluene is used in embodiments of the invention, subsequent alcohol washes are eliminated and replaced with a non-hazardous aqueous wash solution.

The non-polar organic solvent in the composition is typically from about 5% to about 50% by volume of the dewaxing solvent composition. Below the lower percent limit of non-polar organic solvent the dewaxing capability of the composition is significantly decreased. Above the upper limit of non-polar solvent an adverse affect on detergent solubility or water solubility occurs, which adversely affects the effectiveness of a subsequent aqueous wash. The upper limit of organic solvent can be selected among the upper limit values of 50%, 70%, and 75% of the dewaxing solvent composition, while the lower limit of organic solvent can be selected from the lower limit values of 15%, 25% and 35%, to obtain a variety of ranges for embodiments of the invention. Preferably the amount is from about 30% to about 60%, more preferably from about 35% to about 50% of the dewaxing solvent composition.

Because these compositions are typically prepared by combining components without a precise determination of the final volume of the or accounting for volume changes upon mixing, the percentages for each component are qualified with the term “about”, with the understanding that one skilled in the art would appreciate the imprecision of the values as a consequence of composition preparation; however, preferably percentage values are taken to mean their precise value when volume changes upon mixing are accounted for.

The polar organic solvent serves the purpose of dissolving the non-polar solvent, surfactant and optionally water. The polar organic solvent is soluble in water to the extent of at least 1 g per 100 g water, preferably 5 g per 100 g water, more preferably 10 g per 100 g water and most preferably the polar organic solvent is miscible with water. Polar organic solvents include ketones and lower alcohols, which include polyhydroxy alcohols and glycols, and lower ethers. Preferred alcohols are C1 to C5 alcohols. Most preferred are ethanol, ethylene glycol, isopropanol, propylene glycol and mixtures thereof. A preferred ketone solvent is typically C3 to C5 ketone. Most preferred ketone solvents are acetone and methyl ethyl ketone. Preferred ethers are C2 to C6 ethers.

Particularly preferred polar organic solvents are selected from the group consisting of methanol, ethanol, isopropanol, butanol, tert-butanol, allyl alcohol, acetone, ethylene glycol and propylene glycol, and a mixture thereof. Acetonitrile and dimethylformamide are less preferred polar organic solvents. Furthermore, the polar organic solvent can be a mixture of polar organic solvents.

The polar organic solvent in the composition is typically from about 35% to about 50% by volume of the composition. However, the upper limit of polar solvent can be selected among the upper limit values of 50%, 70%, and 75% of the dewaxing solvent composition, while the lower limit of polar solvent can be selected from the lower limit values of 15%, 25% and 35% of the dewaxing solvent composition, to obtain a variety of ranges for embodiments of the invention. Preferably the amount is from about 30% to about 60%, more preferably from about 35% to about 50%. At what combination of components a composition is miscible or separates can readily be determined from a phase diagram showing phase separation for different relative amounts of the components of the solution/mixture.

Surfactants which find use in the present invention include cationic surfactants, anionic surfactants, non-ionic surfactants, and zwitterionic surfactants. A number of biological detergents (surfactants) are listed as such by Sigma Chemical Company in its catalog of Biochemicals and Reagents Life Science Research. The surfactant serves the purpose of a detergent since it has both hydrophilic and hydrophobic properties. A surfactant for use in the invention is soluble in the solvent used in a composition of the invention. Preferred surfactants are detergents that are soluble in water, ethanol and acetone. Most preferred are those that do not substantially interfere with downstream histochemical analyses, which can be determined, for example, by immunostaining using a solution containing the surfactant.

Surfactants that can be used in compositions of the invention include cationic surfactants of the formula:

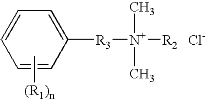

wherein R1 is methyl, ethyl or propyl or isopropyl where n is 1 or 2; R2 is an alkyl group selected from C8H17 to C30H61 orbenzyl group; and R3 is (CH2)m, where m is from 1 to 10, or R3 is (OCH2CH2)p where p is from 1 to 10. Cationic surfactants of this formula are soluble in the polar organic solvents. Many preferred embodiments of the invention contain the cationic surfactant benzalkonium chloride or benzethonium chloride. Additional cationic detergents, not necessarily of this formula, include dodecyltrimethylammonium bromide, benzyldimethylhexadecyl ammonium chloride, cetylphyridinium chloride, methylbenzethonium chloride, and 4-picoline dodecyl sulfate.

Other surfactants that can be used in the compositions of the invention include anionic surfactants having the formula:

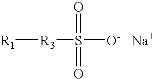

wherein R1 is C6H13 to C30H61, and R3 is O, CH2 or phenyl group. Anionic surfactants of this formula are soluble in polar organic solvent. Examples of anionic detergents, not necessarily having this formula, include alginic acid, caprylic acid, cholic acid, 1-decanesulfonic acid, deoxycholic acid, 1-dodecanesulfonic acid, N-lauroylsarcosine, and taurocholic acid. Other anionic synthetic non-soap detergents, which are represented by the water-soluble salts of organic sulfuric acid reaction products, have in their molecular structure an alkyl radical containing from about 8 to 22 carbon atoms and a radical selected from the group consisting of sulfonic acid and sulfuric acid ester radicals. Examples of these are the sodium or potassium alkyl sulfates, derived from tallow or coconut oil; sodium or potassium alkyl benzene sulfonates; sodium alkyl glyceryl ether sulfonates; sodium coconut oil fatty acid monoglyceride sulfonates and sulfates; sodium or potassium sales of sulfuric acid esters of the reaction product of one mole of a higher fatty alcohol and about 1 to 6 moles of ethylene oxide per molecule and in which the alkyl radicals contain from 8 to 12 carbon atoms; the reaction product of fatty acids esterified with isethionic acid and neutralized with sodium hydroxide, sodium or potassium salts of fatty acid amide of a methyl tauride; and sodium and potassium salts of SO3-sulfonated C10-C24 α-olefins.

Further surfactants that can be used in compositions of the invention include non-ionic surfactants having the formula:

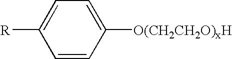

wherein R is a linear or branched C1 to C10 alkyl group and X is an integer from 5 to 40. Most preferably R is

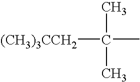

Non-ionic surfactants of this formula are soluble in polar organic solvents. Examples of nonionic detergents, not necessarily having this formula, include decanoyl-N-methylglucamide, diethylene glycol monopentyl ether, n-dodecyl β-D-glucopyranoside, polyoxyethylene ethers of fatty acids (particularly C12-C20 fatty acids, (e.g., sold under the trade name Triton), ethylene oxide condensates of fatty alcohols e.g. sold under the trade name Lubrol), polyoxyethylene sorbitan fatty acid ethers (e.g., sold under the trade name Tween), and sorbitan fatty acid ethers (e.g., sold under the trade name Span).

Nonionic synthetic detergents made by the condensation of alkaline oxide groups with an organic hydrophobic compound. Typical hydrophobic groups include condensation products of propylene oxide with propylene glycol, alkyl phenols, condensation product of propylene oxide and ethylene diamine, aliphatic alcohols having 8 to 22 carbon atoms, and amides of fatty acids. Also nonionic detergents such as amine oxides, phosphine oxides and sulfoxides having semipolar characteristics and be removed.

Specific examples of long chain tertiary amine oxides include dimethyldodecylamine oxide and bis-(2-hydroxyethyl)dodecylamine. Specific examples of phosphine oxides are found in U.S. Pat. No. 3,304,263, and include dimethyldodecylphosphine oxide and dimethyl-(2-hydroxydodecyl) phosphine oxide. A preferred non-ionic detergent surfactant is Triton X-100, which is a trade name for a polyoxyethylene ether of fatty acids (particularly C12-C20 fatty acids).

Zwitterionic surfactants include known compounds of the formula N-alkyl-N,N,-dimethyl-3-ammonio-1-propanesulfonate. Examples of zwitterionic detergents include 3-[3-cholamidopropyl)-dimethylarnmonio]-1]propanesulfonate (commonly abbreviated CHAPS), 3-[cholamidopropyl)-dimethylammonio]-2-hydroxy-1-propane sulfonate (generally abbreviated CHAPSO), N-dodecyl-N-dimethyl-3-ammonio-1-propane sulfonate, and lyso-α-phosphatidyl-choline.

The surfactant in the solution of the present invention is typically from about 0.5% to about 20% weight to volume (g/100 mL) of the solution. Below the lower limit of surfactant poor solubility of wax in the composition is observed. The upper limit of surfactant is a factor of the surfactant's solubility limit. The amount of surfactant is preferably from 0.5% to about 30% by weight, more preferably from about 1.0% to about 25% surfactant by weight, most preferably from about 5% to about 20% by weight of the solution.

Compositions of the invention can also contain water. Most preferably the water in a composition is a saturating amount of water. Above this upper limit phase separation of the composition occurs. Typically water is less than or about 10% by volume of the composition. Some embodiments of the invention, for example as exemplified in the Examples, have less than about 7% water, some have from about 0.5% to about 1.5% water, and still others have less than bout 1% water by volume.

Aldehyde Releasing Reagent Composition

As stated previously, the present invention also employs aldehyde releasing reagent compositions as a means of releasing at least a portion of the aldehyde bonds cause by the use of an aldehyde fixing agent in the tissue specimen.

A procedure for restoring antigenicity of formalin-fixed, paraffin embedded tissue sections by heating the tissue in a microwave in a heavy metal solution has been described in Shi, et al., J. Histochen. Cytochem. 39(b):741-48 (1991). Improved methods and reagents have subsequently been developed and are disclosed, for example, in U.S. Pat. No. 5,578,452 (“the '452 Patent”) entitled “Enhancement of immunochemical staining in aldehyde-fixed tissues,” which is incorporated herein by this reference.

In particular, as disclosed in the '452 Patent, there are a number of aldehyde releasing reagents which can release at least a portion of the aldehyde bonds in the tissue specimen. Such reagents include, for example, nucleophilic agents, oxidizing agents, acid-base pairs and organic acids.

Among the useful nucleophiles are preferably basic nucleophiles. An especially preferred nucleophile is hydroxide anion, which is conveniently supplied as an alkali metal hydroxide such as sodium or potassium hydroxide. Other convenient nucleophiles include primary, secondary, or tertiary amines, especially those with minimal steric hindrance to attack, such as piperidine or morpholine. Hydroxylamine and glycine are preferred. Other nucleophiles include thiols such as mercaptoethanol. Yet another nucleophile of interest is azide, e.g. sodium azide (NaN₃). In general, any nucleophile capable of promoting a reverse Mannich reaction will be capable of cleaving at least some protein cross-linkages, as such reagents will also catalyze reversal of other types of reactions caused by formaldehyde. The concentration of the nucleophile may vary widely, with more concentrated solutions acting more quickly. For short exposures, nucleophile concentrations of 0.5M or greater are usually preferred. In the case of NaOH in methanol, concentrations of one-tenth to one-half of saturation (approximately 0.6 to 3M) are preferred in most circumstances. For hydroxylamine, for example in the form of hydroxylamine hydrochloride, or for glycine, a 10% aqueous solution is preferred.

Other effective aldehyde releasing reagents for the practice of the invention are oxidizing agents. Preferred oxidizing agents are hypochlorites and periodates, especially sodium hypochlorite or sodium periodate. Additional oxidizing agents include hydrazine hydrate. Concentration of the oxidizing agent will vary with the reagent. For example, for sodium hypochlorite, an aqueous (v/v) solution in the range of about 0.01% to about 0.005%, preferably 0.005%, is suitable. For sodium periodate, an aqueous (v/v) solution in the range of about 0.1% to about 1.0%, preferably 0.1%, is suitable. And for hydrazine hydrate, an aqueous (v/v) solution in the range of about 2.0% to about 5.0%, preferably 5.0%, is suitable. Treating the sample with the oxidizing agent is believed to break the cross-linkages between the aldehyde and tissue components and converts the released aldehyde into a non-reactive form, for example by converting formaldehyde to formic acid.

Certain acid/base pairs will function as an aldehyde releasing agent within the scope of this invention. The following pairs are exemplary: Trichloroacetic Acid/NaOH, Toluene Sulfonic Acid/NaOH, Citric Acid/NaOH, Oxalic Acid/NaOH and Tartaric Acid/NaOH. However, for simplicity, it has been found that various of the organic acids alone, such as citric acid, can be used as the reagent in the present aldehyde releasing reagent composition. Where citric acid is employed, it will ordinarily be used from a concentrated stock which facilitates the preparation of the solution of the present invention. For example, an 10× stock solution of the aldehyde releasing reagent composition can contain from about 2 mg/mL to about 6 mg/mL of the aldehyde releasing reagent composition, more commonly from about 4 mg/mL to about 5 mg/mL of the aldehyde releasing reagent composition. In addition, the pH of the stock solution will ordinarily be adjusted to a pH range of from about 5.5 to about 7.5, more commonly 6 to 7.

The solvent for the aldehyde releasing reagent solution may be any solvent compatible with and capable of dissolving the aldehyde releasing reagent. Aqueous solutions are possible; and preferable where the aldehyde releasing reagent is an oxidizing agent, an organic acid/base pair or an organic acid alone. Organic solutions are preferable where the aldehyde releasing reagent is a nucleophile because they promote better penetration of the embedding medium. In addition, proteolytic fragments are insoluble in most organic solvents and therefore tend to remain in place on the slide. In the present solution, there is generally sufficient organic solvent as a result of the dewaxing solvent composition component to serve this function without additional adjustment.

In some embodiments of the invention, the dewaxing solvent compositions or the aqueous wash solutions contain buffer, salts or other reagents useful for wax-solubilization, washes, or subsequent histochemical steps, so long as such optional reagents do not interfere with the wax-solubilizing capability of the composition, the efficiency of a washing step, or subsequent histochemical steps. Reagents useful for subsequent processing or histochemical steps include carboxylic acid esters, enzymes such as lipases, and nucleophilic reagents as described in U.S. Pat. No. 5,578,452. Additional optional reagents include anti-microbial agents and stabilizers that increase composition shelf life. Such anti-microbial agents and stabilizers are well known in the field. Such reagents are typically used at extremely small percentages, typically below 0.1%, compared to the main components. Preferred reagents are those that do not interfere with downstream histochemical analyses.

Each of the individual components of the compositions of the invention is either commercially obtainable, is isolated from natural sources using known procedures, or is synthesized according to known procedures. Compositions of the invention are typically prepared by simple mixing of the components in the indicated amounts.

Methods of preparing reactive samples for sectioning via wax-or paraffin-impregnation are generally well known and easily carried out. The technique is quite simple and involves contacting a wax-embedded specimen with a dewaxing solvent composition of the invention to solubilize the wax that impregnates the specimen prior to histochemical analyses. The method optionally comprises a further step of contacting the dewaxed specimen immediately after dewaxing with an aqueous washing composition comprising a detergent to remove residual dewaxing solvent composition.

Although the dewaxing method is typically and conveniently carried out at room temperature, without the need for a temperature-controlled bath, a more precise control of the required time for satisfactory dewaxing and washing is available if temperature-controlled baths are used. Heating decreases processing time. Operable temperatures range from about 5° to the boiling temperature of the solution, preferably from about 15° C. to the boiling temperature of the solution, more preferably from about 25° C. to about 40° C.

Typically the wax-embedded specimen is contacted with a composition of the invention for a time sufficient to solubilize all or part of the wax embedding the specimen. Factors influencing the solubilization time include temperature, thickness of the specimen section and wax composition. Time for any particular specimen type is best determined empirically. However, five minutes of contact is usually sufficient for specimens mounted on microscope slides. A sectioned specimen, typically affixed to a microscope slide, is contacted with a composition of the invention in any number of ways. Preferably, the specimen is immersed in a bath containing the composition, or alternatively an amount of composition sufficient to solubilize the wax can be placed on the specimen such that the specimen is covered by the composition. After sufficient time of contact has elapsed for wax solubilization to occur, the specimen is removed from contact with the composition, and excess composition is removed from the specimen, for example by draining, blotting or blowing. Optionally, a second or even a further dewaxing step or steps are performed, preferably with fresh dewaxing solvent composition, to farther assure removal of wax from the specimen.

The invention decreases or eliminates the requirement of alcohol baths for post-dewaxing washes. Post dewaxing washes are not always required with compositions of the invention. If such a step proves desirable (because of a particularly sensitive staining procedure, for example) the dewaxed specimen can be contacted with an aqueous wash composition of the invention that comprises a detergent. A preferred wash solution comprises a buffer and a detergent. Preferably the detergent is non-ionic. A preferred buffer/detergent wash solution is phosphate buffered saline with about 1% non-ionic surfactant polyoxyethylene ether, such as BRIJ-35 (trade name for the non-ionic surfactant polyoxyethylene glycol dodecyl ether or polyoxyethylene (23) lauryl ether). Typically the amount of detergent is from about 0.1% to about 5% (weight to volume), preferably from about 0.1% to 2%, and most preferably about 1%.

The pH of the wash composition is most preferably neutral to avoid adversely affecting downstream histochemical analyses. The pH can range from about 2 to about 12, preferably from about 5 to about 8, more preferably from about 5.5 to about 7.5, and more preferably 6.0 to about 6.5. A preferred buffer is one that does not interfere with downstream analyses and/or can be readily removed with a subsequent aqueous wash or blowing. Phosphate buffered saline or Tris-containing buffers are examples of preferred buffers. Washing can occur in any number of ways, including immersion in a wash bath, flowing wash solution over the specimen, diffusing or permeating the wash solution throughout the specimen, or blowing. Wash time is best determined empirically; however, five minutes is usually sufficient. Multiple rinses and larger amounts of washing solution can be used to achieve increased removal of dewaxing solution. A single wash is sufficient for most purposes; however, a second wash is preferred if removal is not sufficient. Optionally, the specimen is finally washed or rinsed in water. A water wash of 3 minutes is usually sufficient for the most rigorous conditions. After washing the specimen is then ready for histochemical or other analyses.

The compositions of the invention, including the wash solutions, are compatible with automated staining systems, as described, for example, in U.S. Pat. Nos. 5,439,649 and 5,948,359, the entire contents of which are hereby incorporated by reference. Dewaxed slides can be provided to an automated staining apparatus or an automated staining apparatus can be provided with compositions of the invention to allow automated dewaxing of the slides prior to automated analyses.

Although preferred surfactants and other components used in a dewaxing solution of the invention are those that do not typically interfere with downstream analyses, particularly at the residual levels remaining on the specimen after the wash procedures, methods known in the art may be applied to enhance surfactant (or other component) removal should residual surfactant (or other component) cause problems in downstream analyses. For residual surfactant removal soluble compounds known to bind a surfactant may be included in an aqueous wash solution. For example, cyclodextrins are known to bind certain surfactants (U.S. Pat. No. 5,032,503) and may be included in a wash solution. Protein, such as bovine serum albumin, can be included in a wash solution to bind and remove residual surfactant. In one preferred embodiment, a surfactant that does not interfere with the downstream analyses, but that can displace the residual surfactant, can be used in an aqueous wash solution. This displacing surfactant is preferably easily removed with a water wash. Polyoxyethylene alkyl ether type non-ionic surfactants are a preferred wash surfactant. BRIJ-35 (trade name for polyoxyethylene glycol dodecyl ether) is an example of one such surfactant.

Also provided is a kit for dewaxing the embedding medium from an embedded tissue specimen while simultaneously releasing aldehyde bonds that interfere with the histological reactivity. In this regard, the kit provides a means for improving the ability of a nucleic acid probe to hybridize with a tissue specimen fixed with an aldehyde fixing agent and embedded in an embedding medium, and for improving the histochemical reaction between the probe and the tissue specimen.

The kit typically comprises a first container containing a solution comprising a dewaxing solvent composition for the embedding medium, and an aldehyde releasing reagent composition, a second container containing a denaturation solution which is capable of separating at least a portion of the nucleic acid strands in said tissue specimen at room temperature, a third container containing a hybridization solution which is capable of hybridizing at least a portion of the nucleic acid probe to the tissue specimen at room temperature, and a detection system for the detection of said nucleic acid probe hybridized to said tissue specimen. The containers are typically, though not necessarily, located in a receptacle specifically adapted to hold them. The kit can be a component of a larger kit for histochemical analyses, such as in a kit for use with automated staining devices. Any of the other reagents described herein can be used in the kit in combination with the specified components.

The methods and kits of the invention are suitable for use in a variety of histochemical applications, particularly immunochemical staining using special stains and other classical stains. In situ hybridization with nucleic acid probes is another particularly pertinent use compatible with compositions and methods of the invention.

The present invention minimizes or eliminates the use of certain toxic organic solvents (e.g. xylene, xylene substitutes, alcohols, and the like) in histological laboratories. The compositions and methodology described herein effectively removes paraffin and other waxes residues from tissue sections and has no adverse effect on quality of tissue sections prepared for histochemistry. Application of this releasing methodology can be extended to other applications where removal of paraffin and other waxes from tissue sections are necessary. In preferred embodiments using isoparaffins, the compositions have a very low order of acute toxicity, being practically non-toxic by oral, dermal and inhalation routes. In addition the methods allow a method of releasing that eliminates the use of graded alcohol washes. Accordingly, the embodiments of the present invention meet the need of providing compositions and methods that minimize dangers to the user and minimize the creation of hazardous waste.

The invention now being generally described, the same will be better understood by reference to the following detailed examples, which are provided for illustration and are not to be considered as limiting the invention unless so specified.

Experimental

In the experimental disclosure which follows, all weights are given in grams (g), milligrams (mg), micrograms (μg), nanograms (ng), or picograms (pg), all amounts are given in moles, millimoles (mmol), micromoles (μmol), nanomoles (nmol), picomoles (pmol), or femtomoles (fmol), all concentrations are given as percent by volume (%), proportion by volume (v:v), molar (M), millimolar (mM), micromolar (μM), nanomolar (nM), picomolar (pM), femtomolar (fM), or normal (N), all volumes are given in liters (L), milliliters (mL), or microliters (μL), and linear measurements are given in millimeters (mm), or nanometers (nm) unless otherwise indicated.

Although in situ hybridization using oligonucleotide probes has been performed at room temperature (6-7), most in situ hybridization (ISH) assays are conducted by techniques using heating, in which denaturation and hybridization procedures are carried out at elevated temperatures. The protocols applied in the previously known room temperature assays, however, are long and complicated and some require an overnight hybridization step. In the present invention, a simple and much briefer room temperature ISH protocol is described. Together with the use of the present dewaxing and aldehyde releasing methods (employing, e.g., the reagent NAR-I), the present ISH protocol results in excellent staining and can be used either manually or with an automated staining system.

Materials and Protocols

Nucleic Acid Retrieval solution I (NAR-I; BioGenex Catalog No. HK-873)

Raw Material & Formulation Requirements

| Product/ | | | | | |
|---|---|---|---|---|---|
| Volume | | Raw Material | Vendor | Cat # | Amount |
| NAR-I/1 L | 1 | Antigen | BioGenex | HK-080-9K | 100 mL |
| | | Retrieval Citra | | | |
| | | Plus solution, | | | |
| | | 10X | | | |
| | 2 | EZ Dewax | BioGenex | HK-585-5K | 75 mL |
| | | solution (Ready | | | |
| | | to use) | | | |
| | 3 | Deionized water | N/A | N/A | 825 mL |

Antigen Retrieval Citra Plus solution (10× Stock):

Citrate buffer pH 6.0

BioGenex Laboratories Catalog—No. HKO80

EZ Dewax Solution (Ready to Use):

BioGenex Laboratories Catalog—No. HK585

Denaturation Solution:

Denaturation Solution is a proprietary formulation of BioGenex Laboratories (available as Denaturation Solution I, Catalog No. HK-829) which comprises strong alkali such as sodium hydroxide.

Protein Block:

Protein Block is a proprietary formulation of BioGenex Laboratories (available as Protein Block, Catalog No. HK-112) which comprises normal goat serum, blue food coloring and Common Antibody Diluent (CABD), a proprietary formulation of BioGenex Laboratories comprising potassium phosphate monobasic and dibasic, sodium chloride, Tween 20, bovine serum albumin, and sodium azide in water.

Peroxide Block:

Peroxide Block is a proprietary formulation of BioGenex Laboratories (available as Peroxide Block, Catalog No. HK-111) which comprises hydrogen peroxide and de-ionized water.

Anti-Fluorescein Antibody:

Anti-Fluorescein Antibody is a proprietary formulation of BioGenex Laboratories (available as Link 1, Catalog No. HK-818) which comprises Common Antibody Diluent (CABD) and Anti-Fluorescein antibody. Anti-Fluorescein antibody is diluted with CABD to approximately 1:2000 dilution (dilution may vary based on the titer of the antibody).

Biotinylated Anti-mouse IgG:

Biotinylated Anti-mouse IgG is a proprietary formulation of BioGenex Laboratories (available as Link 2, Catalog No. HK-827) which comprises Biotin-SP-conjugated F(ab′)₂ fragment Goat Anti-Mouse IgG diluted with CABD to approximately 1:200 dilution (dilution may vary based on the titer of the antibody).

Peroxidase-conjugated Streptavidin:

Peroxidase-conjugated Streptavidin is a proprietary formulation of BioGenex Laboratories (available as Super Sensitive HRP Label, Catalog No. HK-330) which comprises Peroxidase-Conjugated Streptavidin (HRP) diluted with RGLAD to approximately 1:200 dilution (dilution may vary based on the titer of HRP), with Anilino-naphthalene Sulfonic Acid (ANS) as a preservative. Rabbit Goat Labeling Antibody Diluent (RGLAD) is a proprietary formulation of BioGenex Laboratories which comprises potassium phosphate monobasic and dibasic, gentamycin sulfate, neomycin sulfate, sodium chloride, thimerosal, and bovine serum albumin in de-ionized water.

Alkaline Phosphatase-conjugated Streptavidin:

Alkaline Phosphatase-conjugated Streptavidin is a proprietary formulation of BioGenex Laboratories (available as Super Sensitive Alkaline Phosphatase Label, Catalog No. HK-331) which comprises Alkaline Phosphatase-Conjugated Streptavidin (AKP) diluted with a solution of magnesium chloride, zinc chloride, and CABD in de-ionized water to 1:200 dilution (dilution may vary based on AKP titer).

DAB Chromogen for HRP:

DAB Chromogen for HRP is a proprietary formulation of BioGenex Laboratories, which comprises Liquid DAB Chromogen (a proprietary formulation of BioGenex Laboratories available as Catalog No. HK-124 comprising 3,3′-Diaminobenzidine, Citric Acid, and Glycerol) in Liquid DAB Substrate Buffer (a proprietary formulation of BioGenex Laboratories available as Catalog No. HK-128 which comprises MOPS, EDTA, and Triton X-100 in deionized water). Before use, hydrogen peroxide (available as Catalog No. HK-126) and Liquid DAB Chromogen are diluted in Liquid DAB Substrate Buffer.

BCIP/NBT Chromogen for AKP:

BCIP/NBT Chromogen for AKP is a proprietary formulation of BioGenex Laboratories (available as BCIP/NBT Chromogen, Catalog No. HK-188) which comprises 5-Bromo-4-Chloro-3′-Indolyl Phosphate (BCIP) and N,N-Dimethyl-Formamide in de-ionized water as the BCIP stock and Nitro-blue Tetrazolium Chloride (NBT) and N,N-Dimethyl-Formamide in de-ionized water as the NBT stock in a substrate buffer comprising AMPD, sodium chloride and MgCl₂.6H₂O in de-ionized water.

Counterstain:

Hematoxylin Counterstain (for the HRP/DAB detection system) is readily available ready-to-use from commercial sources and from BioGenex Laboratories as Catalog No. HK-100.

Light Green Counterstain (for the AKP/BCIP/NBT detection system) is a proprietary formulation of BioGenex Laboratories (available as Light Green Counterstain, Catalog No. HK-722) which comprises Light Green SF Yellow, acetic acid and reagent alcohol in de-ionized water.

Hybridization Solution (Probes are Dissolved in Hybridization Solution):

Hybridization Solution is a proprietary formulation of BioGenex Laboratories (available as Hybridization Solution, Catalog No. HK-881) which comprises sodium chloride, sodium citrate, Tris-HCl, EDTA disodium salt, Dextran sulfate, Nonidet P-40, Ficoll Type 40, polyvinylpyrolidone 40, bovine albumin Fraction V, salmon sperm DNA and formamide in DEPC-treated water.

Power Block:

A special protein block solution is available, for use in Fluorescent in situ hybridization (FISH) procedures, as Power Block, a proprietary formulation of BioGenex Laboratories (available as Power Block, Catalog No. HK-083) which comprises casein sodium, potassium phosphate monobasic and dibasic, sodium chloride, gentamycin, neomycin sulfate, and sodium azide in de-ionized water.

Procedure for Nucleic Acid Retrieval:

(1) Bring NAR-I solution to room temperature and shake to mix well before use.

(2) Fill plastic slide container (200-250 mL) with NAR-I solution.

(3) Microwave slides, in NAR-I, on high power for 3 minutes.

(4) Discard and replace NAR-I, again microwave on high power for 3 minutes.

(5) Keep in same NAR-I and microwave at 30% power for 10 minutes.

(6) Keep in NAR-I and cool at least 30 minutes.

(7) Wash in at least 2 changes of Optimax Wash Buffer.

(8) Place slides on stainer and cover with additional Optimax Wash Buffer.

(9) Proceed with ISH staining protocol.

Fluorescent in Situ Hybridization (FISH) Using NAR

1. Procedure for Nucleic Acid Retrieval as detailed above.

2. Room temperature FISH:

| | | | Number of | Number of |
|---|---|---|---|---|
| | Number of | Incubation | buffer rinses | water rinses |
| Reagent | Incubations | time | after incubation | after incubation |
| Denaturation soln. | 1 | 5-15 min | 3 | 0 |
| Probe (fluorescein | 1 | 15-30 min | 4 | 0 |
| labeled) in | | | | |
| hybridization soln. | | | | |
| 0.2X PBS | 2 | 10 min | 4 | 0 |
| Protein block soln. | 1 | 5 min | 0 | 0 |
| Biotinylated Anti- | 1 | 20 min | 4 | 0 |
| fluorescein Antibody | | | | |
| Streptavidin- | 1 | 20 min | 4 | 0 |
| phycoerythrin | | | | |

Mount the slides with mounting medium containing anti-fade

Room temperature ISH staining protocol using peroxidase-conjugated streptavidin (HRP) detection system:

| | | | Number of | Number of |
|---|---|---|---|---|
| | Number of | Incubation | buffer rinses | water rinses |
| Reagent | Incubations | time | after incubation | after incubation |
| Denaturation | 1 | 5-15 min | 0 | 3 |
| soln. | | | | |
| Probe in | 1 | 15-30 min | 0 | 1 |
| hybridization | | | | |
| soln. | | | | |
| 2XSSC | 2 | 5 min | 0 | 1 |
| 0.5XSSC | 1 | 5 min | 3 | 0 |
| Protein Block | 1 | 20 min | 3 | 0 |
| Peroxide Block | 1 | 20 min | 3 | 0 |
| Anti- | 1 | 20 min | 3 | 0 |
| fluorescein | | | | |
| antibody | | | | |
| Biotinylated | 1 | 20 min | 3 | 0 |
| anti-mouse IgG | | | | |
| Peroxidase- | 1 | 20 min | 3 | 0 |
| conjugated | | | | |
| Streptavidin | | | | |
| DAB | 1 | 5-30 min | 3 | 0 |
| chromogen | | | | |
| Hematoxylin | 1 | 30 sec | 0 | 0 |
| counterstain | | | | |

Room temperature ISH staining protocol using alkaline phosphotase-conjugated streptavidin (AKP) detection system:

| | | | Number of | Number of |
|---|---|---|---|---|
| | Number of | Incubation | buffer rinses | water rinses |
| Reagent | Incubations | time | after incubation | after incubation |
| Denaturation | 1 | 5-15 min | 0 | 3 |
| soln. | | | | |
| Probe in | 1 | 15-30 min | 0 | 1 |
| hybridization | | | | |
| soln. | | | | |
| 2XSSC | 2 | 5 min | 0 | 1 |
| 0.5XSSC | 1 | 5 min | 3 | 0 |
| Protein block | 1 | 20 min | 3 | 0 |
| Anti- | 1 | 20 min | 3 | 0 |
| fluorescein | | | | |
| antibody | | | | |
| Biotinylated | 1 | 20 min | 3 | 0 |
| anti-mouse IgG | | | | |
| Alkaline | 1 | 20 min | 3 | 0 |
| phosphatase- | | | | |
| conjugated | | | | |
| Streptavidin | | | | |
| BCIP/NBT | 1 | 5-30 min | 3 | 0 |
| chromogen | | | | |
| Light green | 1 | 30 sec | 0 | 0 |
| counterstain | | | | |

The procedure for Nucleic Acid Retrieval and the protocol of room temperature ISH using either HRP or AKP detection system were followed. The BioGenex Optimax Automated Staining System was used for ISH staining.

Initial tests of room temperature ISH with Nucleic Acid Retrieval using HPV 6/11, Alu, and EBER probes were performed. These tests were done using the HRP detection system. Subsequent validation tests using HPV 6/11, EBER and Alu probes were performed. These were done primarily with the HRP detection system; certain tests were performed using the AKP detection system.

Using the same concentration of probes, ISH was conducted and compared with or without nucleic Acid Retrieval (NAR) in combination with heating or room temperature (RT) methods. In the method utilizing heating, denaturation was performed by heating the tissue specimen at 95° C. and hybridization was performed at 37° C. In the RT method, denaturation was performed by using the denaturation solution at room temperature and hybridization was performed at room temperature.

Results:

The results are summarized in the following table and indicate that ISH with NAR in combination with the RT method produced the best results:

| | Without NAR | | | |
|---|---|---|---|---|
| | (Proteinase K treatment) | | With NAR | |
| | RT method | Heating method | RT method | Heating method |
| Probe | (signal/background) | (signal/background) | (signal/background) | (signal/background) |
| Alu | 0/0 | 4.0/0 | 4.0/0 | 0/0 |
| | 0/0 | 0/0 | 4.0/0 | 3.0/0 |
| EBER | 4.5/0 | 4.5/0 | 4.5/0 | 4.0/0 |
| | 4.5/0 | 4.5/0 | 4.5/0 | 4.0/0 |
| HPV6/11 | 3.5/0 | 4.0/0 | 4.5/1.0 | 4.0/0.5 |
| | 0/0 | 4.5/0 | 4.5/1.5 | 4.5/1.0 |

Note:
The intensity of the staining was scored from 0 to 4.5, with 4.5 representing the strongest intensity and 0 indicating no staining
fewer positive cells when compared with NAR/RT slides
even fewer positive cells when compared with NAR/RT slides
a few positive cells present

All tissue sections tested gave strong specific signals while the background was minimal. The quality of the staining was much improved using Nuclear Acid Retrieval in combination with room temperature ISH based on signal to background ratio. The probe concentrations required for HPV 6/11, EBER and Alu probes were 25-, 28- and 200-fold less, respectively, than concentrations used in room temperature ISH without Nucleic Acid Retrieval.

All patents and patent applications cited in this specification are hereby incorporated by reference as if they had been specifically and individually indicated to be incorporated by reference.

Although the foregoing invention has been described in some detail by way of illustration and Example for purposes of clarity and understanding, it will be apparent to those of ordinary skill in the art in light of the disclosure that certain changes and modifications may be made thereto without departing from the spirit or scope of the appended claims.

### References

1. Sibony, M., Commo, F., Callard, P. and Gasc, J. -M. Enhancement of mRNA in situ hybridization signal by microwave heating. Lab. Invest., 73:586-591, 1995.

2. Lan, H. Y., Mu, W., Ng, Y. Y., Nikolic-Paterson, D. J. and Atkins, R. C. A simple, reliable, and sensitive method for nonradioactive in situ hybridization: use of microwave heating to improve hybridization efficiency and preserve tissue morphology. J. Histochem. Cytochem., 44:281-287, 1996.

3. Oliver, K. R., Heavens, R. P. and Sirinathsingh, J. I. Quantitative comparison of pretreatment regimens used to sensitize in situ hybridization using oligonucleotide probes on paraffin-embedded brain tissue. J Histochem. Cytochem., 45:1701-1714, 1997.

4. Wilkens, L., von Wasielewski, R., Werner, M., Nolte, M. and Georgii, A. Microwave preteatment improves RNA-ISH in various formalin-fixed tissues using a uniform protocol. Path. Res. Pract., 192:588-594, 1996.

5. Shelton, J. M., Lee, M. -H., Richardson, J. A. and Patel, S. B. Microsomal triglyceride transfer protein expression during mouse development. J. Lipid. Res., 41:532-537, 2000.

6. Arentzen, R., Baldino, F., Jr., Davis, L. G., Higgins, G. A., Lin, Y., Manning, R. W. and Wolfson, B. In situ hybridization of putative somatostatin mRNA within hypothalamus of the rat using synthetic oligonucleotide probes. J. Cell. Biochem., 27:415-422, 1985.

7. Department of Cytochemistry and Cytometry, University of Leiden, The Netherlands. Detection of neuropeptide mRNAs in tissue sections using oligonucleotides tailed with fluorescein-12-dUTP or DIG-dUTP. In: Roche Molecular Biochemicals (ed) Nonradioactive in situ hybridization application manual, 2nd edition, pp. 146-147, 1996.

1. A method for improving the histochemical reactivity of a tissue specimen fixed with an aldehyde fixing agent and embedded in an embedding medium comprising:
(a) contacting said tissue specimen with a solution comprising
i) a dewaxing solvent composition for said embedding medium, and
ii) an aldehyde releasing reagent composition;
(b) heating said solution and said tissue specimen;
(c) maintaining said heating for a time sufficient to solubilize at least a portion of said embedding medium and to release at least a portion of the aldehyde bonds fixing said tissue specimen; and
(d) performing a histochemical reaction on said tissue specimen.

2. The method as recited in claim 1 wherein said dewaxing solvent composition comprises a mixture of a non-polar organic solvent, a polar organic solvent and a surfactant in amounts sufficient to release a sufficient portion of the embedding medium associated with the tissue specimen to permit histochemical analysis without substantial adverse effect on the histochemical reactivity of the tissue specimen.

3. The method as recited in claim 1, wherein said dewaxing solvent composition comprises:
(a) about 30% to about 50% by volume of at least one organic solvent selected from the group consisting of aromatic hydrocarbons, terpenes and isoparaffinic hydrocarbons;
(b) about 30% to about 50% by volume of at least one water soluble polar organic solvent;
(c) about 0.5% to about 20% by weight to volume of at least one water soluble surfactant; and
(d) sufficient water to complete the volume of the dewaxing solvent composition.

4. The method as recited in claim 2, wherein the organic solvent is limonene.

5. The method as recited in claim 2, wherein the organic solvent comprises an isoparaffinic hydrocarbon.

6. The method as recited in claim 2, wherein the organic solvent is an alkylbenzene or dialkylbenzene.

7. The method as recited in claim 2, wherein the polar organic solvent is selected from the group consisting of C-1 to C-5 alcohols, C-3 to C-5 ketones, and C-2 to C-6 ethers.

8. The method as recited in claim 7, wherein the polar organic solvent comprises at least one solvent selected from the group consisting of methanol, ethanol, propanol, isopropanol, butanol, tert-butanol, acetone, ethylene glycol, allyl alcohol, and propylene glycol.

9. The method as recited in claim 2, wherein the surfactant comprises a cationic or anionic surfactant.

10. The method as recited in claim 9, wherein the surfactant is a cationic surfactant having the formula 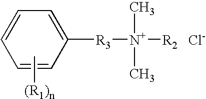
wherein R₁ is methyl, ethyl or propyl or isopropyl where n is 1 or 2; R₂ is an alkyl from C₈H₁₇ to C₃₀H₆₁ or a benzyl group; and R₃ is (CH₂)ₘ where m is from 1 to 10, or R₃ is (OCH₂CH₂)ₚ where p is from 1 to 10.

11. The method as recited in claim 10, wherein the cationic surfactant is benzalkonium chloride or benzethonium chloride.

12. The-method as recited in claim 9, wherein the surfactant is an anionic surfactant having the formula 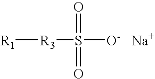
wherein R₁ is C₆H₁₃ to C₃₀H₆₁ and R₃ is CH₂ or a phenyl group.

13. The method as recited in claim 2, wherein the surfactant is a non-ionic surfactant.

14. The method as recited in claim 13, wherein the non-ionic surfactant has the formula 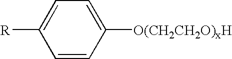
wherein R is a linear or branched C1 to C10 alkyl group and X is from 5 to 40.

15. The method as recited in claim 13, wherein the non-ionic surfactant contains polyoxyethylene ethers of C₁₂ to C₂₀ fatty acids.

16. The method as recited in claim 2 which further comprises the step of washing said specimen after said contacting step with an aqueous wash solution comprising a detergent under conditions sufficient to remove at least a portion of any residual dewaxing solvent composition from said tissue specimen.

17. The method as recited in claim 1 wherein the aldehyde releasing reagent composition comprises citrate.

18. The method as recited in claim 17 wherein the citrate is provided as citric acid in a concentration of from about 2 mg/mL to about 6 mg/mL of said aldehyde releasing reagent composition.

19. The method as recited in claim 17 wherein the citrate is provided as citric acid in a concentration of from about 4 mg/mL to about 5 mg/mL of said aldehyde releasing reagent composition.

20. The method as recited in claim 17 wherein the aldehyde releasing reagent composition is at a pH of from about 5.5 to about 7.5.

21. The method as recited in claim 1 wherein said solution comprises:
(a) about 3% to about 25% by volume of said dewaxing solvent composition;
(b) about 2% to about 20% by volume of said aldehyde releasing reagent composition;
(c) about 45% to about 95% by volume of water.

22. A method as recited in claim 1, wherein steps (b) and (c) are sequentially repeated for a plurality of times sufficient to solubilize at least a portion of said embedding medium and to release at least a portion of the aldehyde bonds fixing said tissue specimen.

23. A method for improving the ability of a nucleic acid probe to hybridize with a tissue specimen which has been fixed with an aldehyde fixing. agent and embedded in an embedding medium comprising:
(a) contacting said tissue specimen with a solution comprising
i) a solvent for said embedding medium, and
ii) an aldehyde releasing reagent;
(b) heating said solution and said tissue specimen;
(c) maintaining said heating for a time sufficient to solubilize at least a portion of said embedding medium and to release at least a portion of the aldehyde fixing agent from said tissue specimen;
(d) exposing said tissue specimen to a denaturation solution at room temperature for a time and under conditions sufficient to allow separation of nucleic acid strands in the tissue;
(e) exposing said tissue specimen to at least one nucleic acid probe at room temperature for a time and under conditions sufficient to allow at least one portion of said nucleic acid probe to hybridize to said tissue specimen; and
(f) exposing said tissue specimen to a series of reagents contained in the peroxidase-conjugated streptavidin (HRP) or alkaline phosphatase-conjugated streptavidin (AKP) detection systems for a time and under conditions sufficient to allow colorimetric detection of said nucleic acid probe.

24. A kit for improving the ability of a nucleic acid probe to hybridize with a tissue specimen fixed with an aldehyde fixing agent and embedded in an embedding medium, and for improving the histochemical reaction between the probe and a probe detection system, comprising:
(a) a first container containing a solution comprising
i) a dewaxing solvent composition for said embedding medium, and
ii) an aldehyde releasing reagent composition; and
(b) a second container containing a denaturation solution which is capable of separating at least a portion of the nucleic acid strands in said tissue specimen at room temperature;
(c) a third container containing a hybridization solution which is capable of hybridizing at least a portion of said nucleic acid probe to said tissue specimen at room temperature; and
(d) a detection system for the detection of said nucleic acid probe hybridized to said tissue specimen.
